(19) United States
(12) Patent Application Publication
(10) Pub. No.: US 2021/0080467 A1
(43) Pub. Date: Mar. 18, 2021

(54) USE OF SK1 AS BIOMARKER FOR PREDICTING RESPONSE TO IMMUNECHECKPOINT INHIBITORS

(71) Applicants: INSERM (INSTITUT NATIONAL DE LA SANTÉ ET DE LA RECHERCHE MÉDICALE), Paris (FR); UNIVERSITÉ PAUL SABATIER TOULOUSE III, Toulouse (FR); CENTRE HOSPITALIER UNIVERSITAIRE DE TOULOUSE, Toulouse (FR)

(72) Inventors: Céline COLACIOS VIATGÉ, Toulouse (FR); Caroline IMBERT, Toulouse (FR); Bruno SEGUI, Toulouse Cedex 1 (FR); Nicolas MEYER, Toulouse (FR); Laurence LAMANT-ROCHAIX, Toulouse (FR); Thierry LEVADE, Toulouse (FR); Nathalie ANDRIEU-ABADIE, Toulouse (FR)

(21) Appl. No.: 16/971,386

(22) PCT Filed: Feb. 20, 2019
(86) PCT No.: PCT/EP2019/054213

§ 371 Date: Aug. 20, 2020

(30) Foreign Application Priority Data
Feb. 21, 2018 (EP) 18305178.8
May 28, 2018 (EP) 18305644.9

(51) Int. Cl. G01N 33/574; G01N 33/533

(52) U.S. Cl. CPC G01N 33/57484; G01N 33/533

(57) ABSTRACT

Immune checkpoint inhibitors (ICI) have revolutionized therapy for advanced cancer, however many patients still do not respond to treatment. However, the efficacy and effectiveness of these therapies varies greatly across individual patients and among different tumour types. A substantial unmet need is thus the development of biomarkers of response to ICI, in order to identify, before initiation of treatment, which patients are likely to experience a response to and clinical benefit from such treatments. Here, the inventors analyzed SPHK1 mRNA in tumor biopsies by in situ hybridization using the RNAscope technology in a cohort of 32 patients suffering from metastatic melanoma. They showed that elevated expression of SPHK1, encoding sphingosine kinase 1 (SK1), which produces the oncometabolite sphingosine-1-phosphate (S1P) is associated with a poor survival in metastatic melanoma patients treated with to the well-known immune-checkpoint inhibitor anti-PD-1 antibody. Accordingly, the present invention relates to the use of SK1 as biomarker for predicting response to immune-checkpoint inhibitors.

### FIELD OF THE INVENTION

The present invention relates to methods for predicting response to immune-checkpoint inhibitors.

### BACKGROUND OF THE INVENTION

Cancer immunotherapy with immune-checkpoint inhibitors (ICI) is based on the inhibition of the tumour-mediated suppression of anticancer immune responses. T-cell activation is indeed regulated by the interplay of the stimulatory and inhibitory ligand-receptor interactions between T cells, dendritic cells, tumour cells, and macrophages in the tumour microenvironment (TME), with tumour cells acting as critical mediators of immunosuppression. Owing to their roles as regulators of T-cell activation, these receptor-ligand pairs are called ‘immune checkpoints’. Agents targeting these checkpoints have been identified as promising treatment options for patients with cancer. Immune-checkpoint inhibitors (ICIs) include, among others, monoclonal antibodies to the receptor cytotoxic T-lymphocyte antigen-4 (CTLA-4) expressed on T cells; programmed cell death protein 1 (PD-1), also expressed on T cells; or the PD-1 ligand (PD-L1), which is expressed by a variety of cell types, including some tumour cells. For instance, the anti-PD-1 antibodies nivolumab and pembrolizumab, and the anti-PD-L1 antibody atezolizumab, have shown marked therapeutic activity in various solid tumours and lymphomas, resulting in a number of regulatory approvals of these agents for the treatment of different malignancies. However, the efficacy and effectiveness of these therapies varies greatly across individual patients and among different tumour types. A substantial unmet need is thus the development of biomarkers of response to ICI, in order to identify, before initiation of treatment, which patients are likely to experience a response to and clinical benefit from such treatments.

The SK type 1 isoform (SK1), which is overexpressed in numerous human tumors including melanoma, leads to increased levels of sphingosine-1-phosphate (S1P) (8, 9) that is a well-known oncometabolite. The SK1/S1P axis could modulate different hallmarks of cancer such as cell proliferation, cell death, metastasis and angiogenesis (10, 11). Moreover, S1P is a well-known regulator of lymphocyte trafficking and differentiation under different pathophysiological conditions (12, 13). However, the impact of high expression levels of SK1 in melanoma cells on the function and phenotype of tumor-infiltrating lymphocytes (TILs) is not documented. Moreover, the role of SK1 as a biomarker for predicting the response to ICI has not yet been documented.

### SUMMARY OF THE INVENTION

The present invention relates to methods for predicting response to immune-checkpoint inhibitors. In particular, the present invention is defined by the claims.

### DETAILED DESCRIPTION OF THE INVENTION

Immune checkpoint inhibitors (ICI) have revolutionized therapy for advanced cancer, however many patients still do not respond to treatment. However, the efficacy and effectiveness of these therapies varies greatly across individual patients and among different tumour types. A substantial unmet need is thus the development of biomarkers of response to ICI, in order to identify, before initiation of treatment, which patients are likely to experience a response to and clinical benefit from such treatments. Here, the inventors showed that elevated expression of sphingosine kinase 1 (SK1), which produces the oncometabolite sphingosine-1-phosphate (S1P) is associated with a poor survival in metastatic melanoma patients treated with to the well-known immune-checkpoint inhibitor anti-PD-1 antibody.

Accordingly, the first object of the present invention relates to a method for determining whether a patient suffering from a cancer will achieve a response with an immune checkpoint inhibitor comprising i) determining the expression level of SK1 in a tumor sample obtained from the patient, ii) comparing the expression level determined at step i) with a predetermined reference value and iii) concluding that the patient will not achieve a response when the level determined at step i) is higher than the predetermined reference value or concluding that the patient will achieve a response when the level determined at step i) is lower than the predetermined reference value.

As used herein, the term “patient” denotes a mammal, such as a rodent, a feline, a canine, and a primate. Particularly, the patient according to the invention is a human. Particularly, the patient according to the invention has or is susceptible to have cancer.

As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the patient suffers from melanoma. As used herein, “melanoma” refers to a condition characterized by the growth of a tumor arising from the melanocytic system of the skin and other organs. Most melanocytes occur in the skin, but are also found in the meninges, digestive tract, lymph nodes and eyes. When melanoma occurs in the skin, it is referred to as cutaneous melanoma. Melanoma can also occur in the eyes and is called ocular or intraocular melanoma. In some embodiments, the patient suffers from a metastatic melanoma.

The method is thus particularly suitable for discriminating responder from non responder. As used herein the term “responder” in the context of the present disclosure refers to a patient that will achieve a response, i.e. a patient where the cancer is eradicated, reduced or improved. According to the invention, the responders have an objective response and therefore the term does not encompass patients having a stabilized cancer such that the disease is not progressing after the immune checkpoint therapy. A non-responder or refractory patient includes patients for whom the cancer does not show reduction or improvement after the immune checkpoint therapy. According to the invention the term “non responder” also includes patients having a stabilized cancer. Typically, the characterization of the patient as a responder or non-responder can be performed by reference to a standard or a training set. The standard may be the profile of a patient who is known to be a responder or non responder or alternatively may be a numerical value. Such predetermined standards may be provided in any suitable form, such as a printed list or diagram, computer software program, or other media. When it is concluded that the patient is a non responder, the physician could take the decision not to prescribed immune checkpoint therapy to avoid any further adverse sides effects.

As used herein, the term “immune checkpoint inhibitor” has its general meaning in the art and refers to any compound inhibiting the function of an immune inhibitory checkpoint protein (see Table A). Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules.

TABLE A
examples of genes encoding for immune checkpoint proteins:

| | | GENE |
|---|---|---|
| Gene | Name | ID |
| IDO1 | indoleamine 2,3-dioxygenase 1 | 3620 |
| CD40 | CD40 molecule, TNF receptor superfamily | 958 |
| | member 5 | |
| CD274 | CD274 molecule, also known as B7-H; B7H1; | 29126 |
| | PDL1; PD-L1; PDCD1L1; PDCD1LG1 | |
| ICOS | inducible T-cell co-stimulator | 29851 |
| TNFRSF9 | tumor necrosis factor receptor superfamily | 3604 |
| | member 9, also known as ILA; 4-1BB; CD137; | |
| | CDw137 | |
| TNFRSF18 | tumor necrosis factor receptor superfamily | 8784 |
| | member 18, also known as AITR; GITR; CD357; | |
| | GITR-D | |
| LAG3 | lymphocyte-activation gene 3 | 3902 |
| IL2RB | interleukin 2 receptor, beta | 3560 |
| HAVCR2 | hepatitis A virus cellular receptor 2 | 84868 |
| TNFRSF4 | tumor necrosis factor receptor superfamily | 7293 |
| | member 4 | |
| CD276 | CD276 molecule | 80381 |
| CTLA4 | cytotoxic T-lymphocyte-associated protein 4 | 1493 |
| PDCD1LG2 | programmed cell death 1 ligand 2, also known as | 80380 |
| | B7DC; Btdc; PDL2; CD273; PD-L2; PDCD1L2; | |
| | bA574F11.2 | |
| VTCN1 | V-set domain containing T cell activation | 79679 |
| | inhibitor 1, also known as B7H4 | |
| PDCD1 | programmed cell death 1, also known as PD1; | 5133 |
| | PD-1; CD279; SLEB2; hPD-1; hPD-1; hSLE1 | |
| BTLA | B and T lymphocyte associated | 151888 |
| CD28 | CD28 molecule | 940 |
| C10orf54 | chromosome 10 open reading frame 54 | 64115 |
| CD27 | CD27 molecule | 939 |

As used herein, the term “immune checkpoint inhibitor” refers to molecules that totally or partially reduce, inhibit, interfere with or modulate one or more immune checkpoint proteins.

As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al. 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein Inhibition includes reduction of function and full blockade. In some embodiments, the immune checkpoint inhibitor could be an antibody, synthetic or native sequence peptides, small molecules or aptamers which bind to the immune checkpoint proteins and their ligands.

Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an antibody selected from the group consisting of anti-CTLA4 antibodies (e.g. Ipilimumab), anti-PD1 antibodies, anti-PDL1 antibodies, anti-TIM-3 antibodies, anti-LAG3 antibodies, anti-B7H3 antibodies, anti-B7H4 antibodies, anti-BTLA antibodies, and anti-B7H6 antibodies.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (also called Opdivo®, MDX-1106-04, ONO-4538, BMS-936558), Pembrolizumab (also called Lambrolizumab, KEYTRUDA® or MK-3475, MERCK) and Pidilizumab (also known as CT-011, hBAT, and hBAT-1). In some embodiments, the PD-1 binding antagonist is AMP-224 (also known as B7-DCIg).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

As used herein, the term “TIM-3” has its general meaning in the art and refers to T cell immunoglobulin and mucin domain-containing molecule 3. The natural ligand of TIM-3 is galectin 9 (Gal9). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophenyl)-N′-hydroxy-4-{[2-(sulfamoylamino)-ethyl]amino}-1,2,5-oxadiazole-3 carboximidamide:

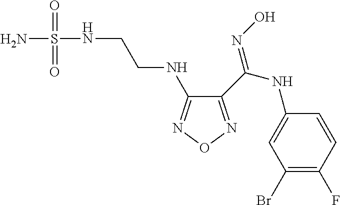 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

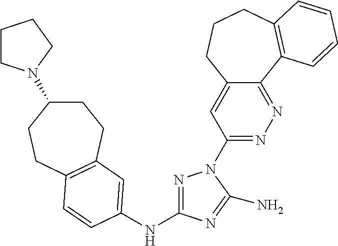 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

As used herein, the term “tumor sample” means any tumor sample derived from the patient. In some embodiments, the sample is obtained before any therapy with an immune checkpoint inhibitor. Said tissue sample is obtained for the purpose of the in vitro evaluation. In some embodiments, the tumor sample may result from the tumor resected from the patient. In some embodiments, the tumor sample may result from a biopsy performed in the primary tumor of the patient or performed in metastatic sample distant from the primary tumor of the patient. In some embodiments, the tumor sample is a sample of circulating tumor cells. As used herein, the term “circulating tumor cell” or “CTC” refers to a cancer cell derived from a cancerous tumor that has detached from the tumor and is circulating in the blood stream of the patient. Typically the CTCs are isolated from the blood sample using a filter and/or a marker based method. For example, CTCs can be isolated using an anti-EpCAM antibody to magnetically capture CTCs expressing this antigen on their surfaces with for example the CellSearchR system (Scher et al., 2005; Berthold et al., 2008; Madan et al., 2011; Fleming et al., 2006; Gulley and Drake, 2011; Bubley et al., 1999; Scher et al., 2008). Other approaches include for example detecting the presence of circulating nucleic acids (Schwarzenbach et al., 2011), on immunohistochemistry with anti-cytokeratin 8 and 18 antibodies that are also used in combination with the anti-EpCAM antibodies, or on CTC-chips as well as the EPISPOT test, which depletes CD45 cells first and examines the remaining cells. In addition, collagen adhesion matrix assays (CAM assays) can be used (for a review on these methods, see Doyen et al., 2011).

As used herein, the term “sphingosine kinase-1” or “SK1” refers to an enzyme that catalyzes the transformation of sphingosine to sphingosine-1-phosphate (SIP), i.e., phosphorylates sphingosine into SIP. Properties and activities of SK1, e.g., protein sequence of SK1, structural properties of SK1, biochemical properties of SK1, and regulation of SK1, are described in Taha et al. (2006, Journal of Biochemistry and Molecular Biology, 39(2): 113-131). An exemplary human amino acid sequence is represented by SEQ ID NO:1 and an exemplary human nucleic acid sequence is represented by SEQ ID NO:2.

(NCBI reference sequence NP_001136073):
SEQ ID NO: 1
MDPAGGPRGV LPRPCRVLVL LNPRGGKGKA LQLFRSHVQP LLAEAEISFT LMLTERRNHA
RELVRSEELG RWDALVVMSG DGLMHEVVNG LMERPDWETA IQKPLCSLPA GSGNALAASL
NHYAGYEQVT NEDLLTNCTL LLCRRLLSPM NLLSLHTASG LRLFSVLSLA WGFIADVDLE
SEKYRRLGEM RFTLGTFLRL AALRTYRGRL AYLPVGRVGS KTPASPVVVQ QGPVDAHLVP
LEEPVPSHWT VVPDEDFVLV LALLHSHLGS EMFAAPMGRC AAGVMHLFYV RAGVSRAMLL
RLFLAMEKGR HMEYECPYLV YVPVVAFRLE PKDGKGVFAV DGELMVSEAV
QGQVHPNYFW MVSGCVEPPP SWKPQQMPPP EEPL
(NCBI reference sequence NM_001142601.1
SEQ ID NO: 2
AGTGCCCTCC CCGCTCCGCG GCGCCGGCTG CGAAGTTGAG CGAAAAGTTT
GAGGCCGGAG GGAGCGAGGC CGGGGAGTCC GCTCCAGCGG GGCGCTCCAG
TCCCTCAGAC GTGGGCTGAG CTTGGGACGA GCTGCGTTCC GCCCCAGGCC
ACTGTAGGGA ACGGCGGTGG CGCCTCCCCA GCAAACCGGA CCGACTGGGT
CCAGCCGCCG CAGGGAATGA CGCCGGTGCT CCTGCAGCCA CGGCTCCGGG
CGGGGAAGGC GAGCCCCACA GCCGGCCCTG CGACGCCCGC CTGGGCAGCA
CCGATAAGGA GCTGAAGGCA GGAGCCGCCG CCACGGGCAG CGCCCCCACA
GCGCCAGGGA CCCCCTGGCA GCGGGAGCCG CGGGTCGAGG TTATGGATCC
AGCGGGCGGC CCCCGGGGCG TGCTCCCGCG GCCCTGCCGC GTGCTGGTGC
TGCTGAACCC GCGCGGCGGC AAGGGCAAGG CCTTGCAGCT CTTCCGGAGT
CACGTGCAGC CCCTTTTGGC TGAGGCTGAA ATCTCCTTCA CGCTGATGCT CACTGAGCGG
CGGAACCACG CGCGGGAGCT GGTGCGGTCG GAGGAGCTGG GCCGCTGGGA
CGCTCTGGTG GTCATGTCTG GAGACGGGCT GATGCACGAG GTGGTGAACG
GGCTCATGGA GCGGCCTGAC TGGGAGACCG CCATCCAGAA GCCCCTGTGT
AGCCTCCCAG CAGGCTCTGG CAACGCGCTG GCAGCTTCCT TGAACCATTA TGCTGGCTAT
GAGCAGGTCA CCAATGAAGA CCTCCTGACC AACTGCACGC TATTGCTGTG
CCGCCGGCTG CTGTCACCCA TGAACCTGCT GTCTCTGCAC ACGGCTTCGG GGCTGCGCCT
CTTCTCTGTG CTCAGCCTGG CCTGGGGCTT CATTGCTGAT GTGGACCTAG AGAGTGAGAA
GTATCGGCGT CTGGGGGAGA TGCGCTTCAC TCTGGGCACC TTCCTGCGTC TGGCAGCCCT
GCGCACCTAC CGCGGCCGAC TGGCCTACCT CCCTGTAGGA AGAGTGGGTT
CCAAGACACC TGCCTCCCCC GTTGTGGTCC AGCAGGGCCC GGTAGATGCA CACCTTGTGC
CACTGGAGGA GCCAGTGCCC TCTCACTGGA CAGTGGTGCC CGACGAGGAC
TTTGTGCTAG TCCTGGCACT GCTGCACTCG CACCTGGGCA GTGAGATGTT TGCTGCACCC
ATGGGCCGCT GTGCAGCTGG CGTCATGCAT CTGTTCTACG TGCGGGCGGG AGTGTCTCGT
GCCATGCTGC TGCGCCTCTT CCTGGCCATG GAGAAGGGCA GGCATATGGA
GTATGAATGC CCCTACTTGG TATATGTGCC CGTGGTCGCC TTCCGCTTGG AGCCCAAGGA
TGGGAAAGGT GTGTTTGCAG TGGATGGGGA ATTGATGGTT AGCGAGGCCG
TGCAGGGCCA GGTGCACCCA AACTACTTCT GGATGGTCAG CGGTTGCGTG
GAGCCCCCGC CCAGCTGGAA GCCCCAGCAG ATGCCACCGC CAGAAGAGCC
CTTATGACCC CTGGGCCGCG CTGTGCCTTA GTGTCTACTT GCAGGACCCT TCCTCCTTCC
CTAGGGCTGC AGGGCCTGTC CACAGCTCCT GTGGGGGTGG AGGAGACTCC
TCTGGAGAAG GGTGAGAAGG TGGAGGCTAT GCTTTGGGGG GACAGGCCAG
AATGAAGTCC TGGGTCAGGA GCCCAGCTGG CTGGGCCCAG CTGCCTATGT
AAGGCCTTCT AGTTTGTTCT GAGACCCCCA CCCCACGAAC CAAATCCAAA
TAAAGTGACA TTCCCAGCCT GAAAAAAAAA AAAAAAAAA

Determining the expression level of SK1 may be performed by any method well known in the art.

In some embodiments, the determination is performed by immunodetection such as immunohistochemistry (IHC) or immunofluorescence. In some embodiments, a percentage of tumor cells positive for SK1 is determined by IHC. For instance, immunohistochemistry typically includes the following steps i) fixing the tumor tissue sample with formalin, ii) embedding said tumor tissue sample in paraffin, iii) cutting said tumor tissue sample into sections for staining, iv) incubating said sections with the binding partner specific for the marker (i.e. SK1), v) rinsing said sections, vi) incubating said section with a secondary antibody typically biotinylated and vii) revealing the antigen-antibody complex typically with avidin-biotin-peroxidase complex. Accordingly, the tumor tissue sample is firstly incubated the binding partners. After washing, the labeled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems. For example, one or more labels can be attached to the antibody, thereby permitting detection of the target protein (i.e the marker). Exemplary labels include radioactive isotopes, fluorophores, ligands, chemiluminescent agents, enzymes, and combinations thereof. In some embodiments, the label is a quantum dot. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. ³H, ¹⁴C, ³²P, ³⁵S or ¹²⁵I) and particles (e.g. gold). The resulting stained specimens may be imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the marker in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549).

In some embodiments, determining the expression level of SK1 is determined by detecting the quantity of mRNA encoding for SK1. Methods for determining the quantity of mRNA are well known in the art. For example, the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA). Typically, the nucleic acid probes include one or more labels, for example to permit detection of a target nucleic acid molecule using the disclosed probes. Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials. Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH). Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. J. Pathol. 157:1467-1472, 2000 and U.S. Pat. No. 6,942,970. Additional detection methods are provided in U.S. Pat. No. 6,280,929.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the selected peptide in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

It should be noted that the predetermined reference value is not necessarily the median value of expression levels of the gene. Thus in some embodiments, the predetermined reference value thus allows discrimination between a poor and a good prognosis for a patient. In some embodiments, the predetermined reference level correlates with the survival time of the patient and can thus be determined as follows. For example the expression level of the gene has been assessed for 100 samples of 100 subjects. The 100 samples are ranked according to the expression level of the gene. Sample 1 has the highest level and sample 100 has the lowest level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding subject, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the expression level of the gene corresponding to the boundary between both subsets for which the p value is minimum is considered as the predetermined reference value. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the expression level of the gene with the range of values which are identified. In some embodiments, a cut-off value thus consists of a range of quantification values, e.g. centered on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. For example, a patient may be assessed by comparing values obtained by measuring the expression level of the gene, where values higher than 5 reveal that the patient will not achieve a response and values less than 5 reveal that the patient will achieve a response. In some embodiments, a patient may be assessed by comparing values obtained by measuring the expression level of the gene and comparing the values on a scale, where values above the range of 4-6 indicate that the patient will not achieve a response and values below the range of 4-6 indicate that the patient will achieve a response, with values falling within the range of 4-6 indicating an uncertainty about the response.

In some embodiments, step ii) consisting in determining the percentage of tumor cells positive for the expression of SK. In some embodiments, the predetermined reference value thus represents a percentage of tumor cells positive for SK1. In some embodiments, the predetermined reference value is 0, 1, 2, 5, 10, 20, 30, 40 or 50% of positive tumor cells and thereby, levels higher than these values indicate the patient will not achieve a response with the immune checkpoint inhibitor and levels lower than theses values indicate that the patient will achieve a response.

In a particular embodiment, the method according to the invention further comprises a step of classification of subject by an algorithm and determining whether a subject will achieve a response to an immune checkpoint inhibitor treatment.

Typically, the method of the present invention comprises a) quantifying the level of the SK1 in a tumor sample; b) implementing a classification algorithm on data comprising the quantified of SK1 levels so as to obtain an algorithm output; c) determining the probability that the subject will achieve or not a response to an immune checkpoint inhibitor from the algorithm output of step b).

In some embodiments, the method according to the invention wherein the algorithm is selected from Linear Discriminant Analysis (LDA), Topological Data Analysis (TDA), Neural Networks, Support Vector Machine (SVM) algorithm and Random Forests algorithm (RF).selected from Linear Discriminant Analysis (LDA), Topological Data Analysis (TDA), Neural Networks, Support Vector Machine (SVM) algorithm and Random Forests algorithm (RF).

In some embodiments, the method of the invention comprises the step of determining the subject response using a classification algorithm. As used herein, the term “classification algorithm” has its general meaning in the art and refers to classification and regression tree methods and multivariate classification well known in the art such as described in U.S. Pat. No. 8,126,690; WO2008/156617. As used herein, the term “support vector machine (SVM)” is a universal learning machine useful for pattern recognition, whose decision surface is parameterized by a set of support vectors and a set of corresponding weights, refers to a method of not separately processing, but simultaneously processing a plurality of variables. Thus, the support vector machine is useful as a statistical tool for classification. The support vector machine non-linearly maps its n-dimensional input space into a high dimensional feature space, and presents an optimal interface (optimal parting plane) between features. The support vector machine comprises two phases: a training phase and a testing phase. In the training phase, support vectors are produced, while estimation is performed according to a specific rule in the testing phase. In general, SVMs provide a model for use in classifying each of n subjects to two or more disease categories based on one k-dimensional vector (called a k-tuple) of biomarker measurements per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction (Vapnik, 1998 Statistical Learning Theory. New York: Wiley). Any new observation is then classified as belonging to any one of the categories of interest, based where the observation lies in relation to the hyperplane. When more than two categories are considered, the process is carried out pairwise for all of the categories and those results combined to create a rule to discriminate between all the categories. As used herein, the term “Random Forests algorithm” or “RF” has its general meaning in the art and refers to classification algorithm such as described in U.S. Pat. No. 8,126,690; WO2008/156617. Random Forest is a decision-tree-based classifier that is constructed using an algorithm originally developed by Leo Breiman (Breiman L, “Random forests,” Machine Learning 2001, 45:5-32). The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set. In some embodiments, the score is generated by a computer program.

The algorithm of the present invention can be performed by one or more programmable processors executing one or more computer programs to perform functions by operating on input data and generating output. The algorithm can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

In some embodiment, when it is determined that the patient will achieve a response with the immune checkpoint inhibitor, then after the patient is administered with a therapeutically effective amount of said immune checkpoint inhibitor.

Accordingly a further object of the present invention relates to a method of treating a patient suffering from a cancer comprising i) determining the expression level of SK1 in a tumor sample obtained from the patient, ii) comparing the expression level determined at step i) with a predetermined reference value and (iii) administering to said patient a therapeutically effective amount of said immune checkpoint inhibitor when it is concluded that the patient will achieve a response with the immune checkpoint inhibitor according to the present invention.

Accordingly a further object of the present invention also relates to a method of treating a patient suffering from a cancer comprising i) determining the expression level of SK1 in a tumor sample obtained from the patient, ii) comparing the expression level determined at step i) with a predetermined reference value (iii) concluding that the patient will not achieve a response when the level determined at step i) is higher than the predetermined reference value or concluding that the patient will achieve a response when the level determined at step i) is lower than the predetermined reference value and (iv) administering to said patient a therapeutically effective amount of said immune checkpoint inhibitor when it is concluded that the patient will achieve a response with the immune checkpoint inhibitor.

In a further object, the method according to the present invention, wherein immune checkpoint inhibitor is used for treating the patient identified as a responder to immune checkpoint inhibitor

In one embodiment, the administration may be combined to chemotherapy and/or radiotherapy.

As used herein, the term “chemotherapy” has its general meaning in the art and refers to the treatment that consists in administering to the patient a chemotherapeutic agent. In some embodiments, the chemotherapeutic agent is an immunogenic cell death (ICD) inducer, i.e. a pharmacological compounds that kills malignant cells in a way that they elicit an anticancer immune response.(10-19) Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; and pharmaceutically acceptable salts, acids or derivatives of any of the above.

As used herein, the term “radiation therapy” has its general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In some embodiments, the method of the present invention is particularly suitable in the context of a hypo fractionated radiation therapy. As used herein the term “hypo fractionated radiation therapy” has its general meaning in the art and refers to radiation therapy in which the total dose of radiation is divided into large doses and treatments are given less than once a day.

In some embodiment, when it is determined that the patient will not achieve a response with the immune checkpoint inhibitor, the patient is not administered with the immune checkpoint inhibitor and will typically receive a cure of chemotherapy and/or radiotherapy. In some embodiments, when it is concluded that the patient will not achieve a response with the immune checkpoint inhibitor, the patient may be administered with a therapeutically effective amount of SK1 inhibitor and more particularly with a combination of a SK1 inhibitor and an immune checkpoint inhibitor as disclosed in WO2017129769.

Accordingly a further object of the present invention relates to a method of treating a patient suffering from a cancer comprising i) determining the expression level of SK1 in a tumor sample obtained from the patient, ii) comparing the expression level determined at step i) with a predetermined reference value and (iii) administering to said patient a therapeutically effective amount of a SK1 inhibitor when it is concluded that the patient will not achieve a response with the immune checkpoint inhibitor according to the present invention.

Accordingly a further object of the present invention also relates to a method of treating a patient suffering from a cancer comprising i) determining the expression level of SK1 in a tumor sample obtained from the patient, ii) comparing the expression level determined at step i) with a predetermined reference value (iii) concluding that the patient will not achieve a response when the level determined at step i) is higher than the predetermined reference value or concluding that the patient will achieve a response when the level determined at step i) is lower than the predetermined reference value and (iv) administering to said patient a therapeutically effective amount of a SK1 inhibitor when it is concluded that the patient will not achieve a response with the immune checkpoint inhibitor.

In a further object, the method according to the present invention, wherein SK1 inhibitor is used for treating the patient identified as a non-responder to immune checkpoint inhibitor

As used herein the term “SK1 inhibitor” refers to any compound that is capable to inhibit SK1 expression or activity. SK1 inhibitors are well known to the skilled person. For example, the skilled person may easily identify such inhibitors from the following patent publications: WO2003105840, WO2006138660, WO2010033701, WO2010078247, WO2010127093, WO2011020116, WO2011072791, WO2012069852, WO2013119946, WO2014118556 and WO2014157382. In some embodiments, the SK1 inhibitor is an inhibitor of SK1 expression (antisense oligonucleotide, siRNA . . . ).

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In some embodiments, the treatment consists of administering to the subject a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names.

As used herein the terms “administering” or “administration” refer to the act of injecting or otherwise physically delivering a substance as it exists outside the body (e.g., a SK1 inhibitor and/or an immune checkpoint inhibitor) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of a SK1 inhibitor and/or an immune checkpoint inhibitor for use in a method for the treatment of cancer at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The SK1 inhibitor and/or an immune checkpoint inhibitor as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

Accordingly a further object of the present invention relates to a method of treating a patient suffering from a cancer comprising i) determining the expression level of SK1 in a tumor sample obtained from the patient, ii) comparing the expression level determined at step i) with a predetermined reference value and (iii) administering to said patient a therapeutically effective amount of a SK1 inhibitor in combination with an immune checkpoint inhibitor when it is concluded that the patient will not achieve a response with the immune checkpoint inhibitor according to the present invention.

Accordingly a further object of the present invention also relates to a method of treating a patient suffering from a cancer comprising i) determining the expression level of SK1 in a tumor sample obtained from the patient, ii) comparing the expression level determined at step i) with a predetermined reference value (iii) concluding that the patient will not achieve a response when the level determined at step i) is higher than the predetermined reference value or concluding that the patient will achieve a response when the level determined at step i) is lower than the predetermined reference value and (iv) administering to said patient a therapeutically effective amount of a SK1 inhibitor in combination with an immune checkpoint inhibitor when it is concluded that the patient will not achieve a response with the immune checkpoint inhibitor.

In a further object, the method according to the present invention, wherein i) a SK1 inhibitor and ii) an immune checkpoint inhibitor are used as a combined preparation for treating the patient identified as a non-responder to immune checkpoint inhibitor.

In a particular embodiment, i) a SK1 inhibitor and ii) an immune checkpoint inhibitor as a combined preparation according to the invention for simultaneous, separate or sequential use in the method for treating a cancer in a patient.

As used herein, the term “combination” is intended to refer to all forms of administration that provide a first drug together with a further (second, third . . . ) drug. The drugs may be administered simultaneous, separate or sequential and in any order. According to the invention, the drug is administered to the subject using any suitable method that enables the drug to reach the lungs. In some embodiments, the drug administered to the subject systemically (i.e. via systemic administration). Thus, in some embodiments, the drug is administered to the subject such that it enters the circulatory system and is distributed throughout the body. In some embodiments, the drug is administered to the subject by local administration, for example by local administration to the lungs.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

The invention will be further illustrated by the following figures and examples. However, these examples and figures should not be interpreted in any way as limiting the scope of the present invention.

### FIGURES

FIG. 1. High SPHK1 expression correlates with poor survival of melanoma patients treated with anti-PD-1. (A) SPHK1 expression in human nevi (n=17) compared to primary melanoma (P, n=45) and in primary melanoma (P, n=25) compared to metastatic melanoma (M, n=44) was assessed using the Oncomine database. (B) Percentage of cancer cells positive for SPHK1 mRNA staining in metastatic melanoma tissues of 32 patients prior anti-PD-1 treatment. (C) Representative mRNA staining of low and high SPHK1 expression. Skin (P1-P3) or lymph node (P2-P4) biopsies from patients with metastatic melanoma. Percentages indicate the proportion of cancer cells positive for SPHK1 mRNA staining. Large and small blue lines represent 200 and 20 μm, respectively. (D) Progression-Free Survival and (E) Overall Survival curves of patients with more than 50% of melanoma cells positive for SPHK1 (SPHK1 high) (n=11) or less than 50% (SPHK1 Low) (n=21). Survival times were calculated from the first day of the cycle of anti-PD-1 post biopsy. Statistical significance was determined by log-rank test.

### EXAMPLE

Methods

Patient Cohorts

SPHK1 expression analysis in human nevi and melanomas was assessed in 2 different cohorts from Oncomine (Talantov Clin Cancer Res. 2005 Oct. 15; 11(20):7234-42; Xu Mol Cancer Res. 2008 May; 6(5):760-9).

Patient Survival Analyses

Protocol was approved by “CPP du Sud-Ouest et Outre-Mer IV” (Limoges, France). Informed, signed consents from metastatic melanoma patients were obtained. The major clinicopathologic characteristics and available treatment information of the cohorts are presented in Table B.

All survival times were calculated from the first day of the first cycle of anti-PD-1 therapy. Progression-free survival and overall survival were defined using the following first-event definitions: either relapse or death from any cause for PFS, and death from any cause for OS. Patients still alive were censored at their date of last follow-up. Comparison between groups (low expression vs high expression) was performed using log-rank test.

In Situ mRNA Hybridization

In situ detection of SPHK1 transcripts in Formalin-Fixed, Paraffin-Embedded Tissues was performed using the RNAscope assay with RNAScope 2.5 VS Probe—Hs-SPHK1 and the ACD RNAscope 2.0 Red kit (Advanced Cell Diagnostics). Assay specificity was assessed measuring the signal in positive and negative control samples. Positivity of endothelial cells was used as an intrinsic positive control. Cases with positive intrinsic control and no signal in tumor cells were considered as negative. Quantification was assessed by evaluating the percentage of positive tumor cells blinded to clinical response to treatment.

Results

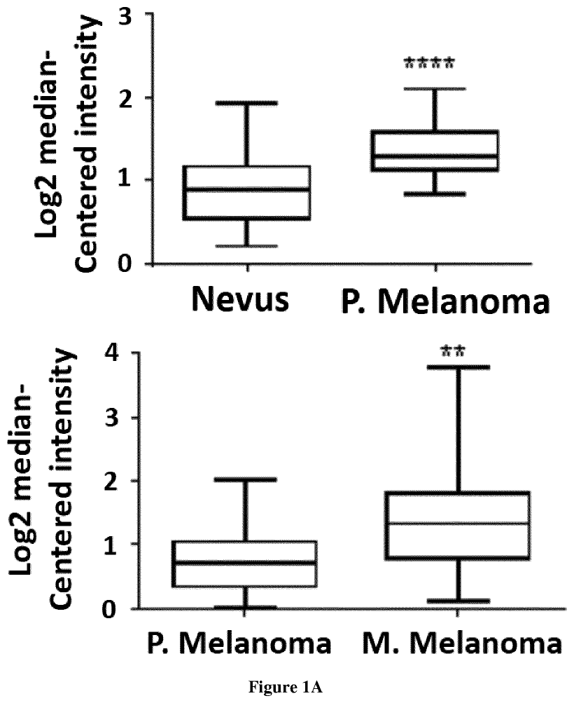

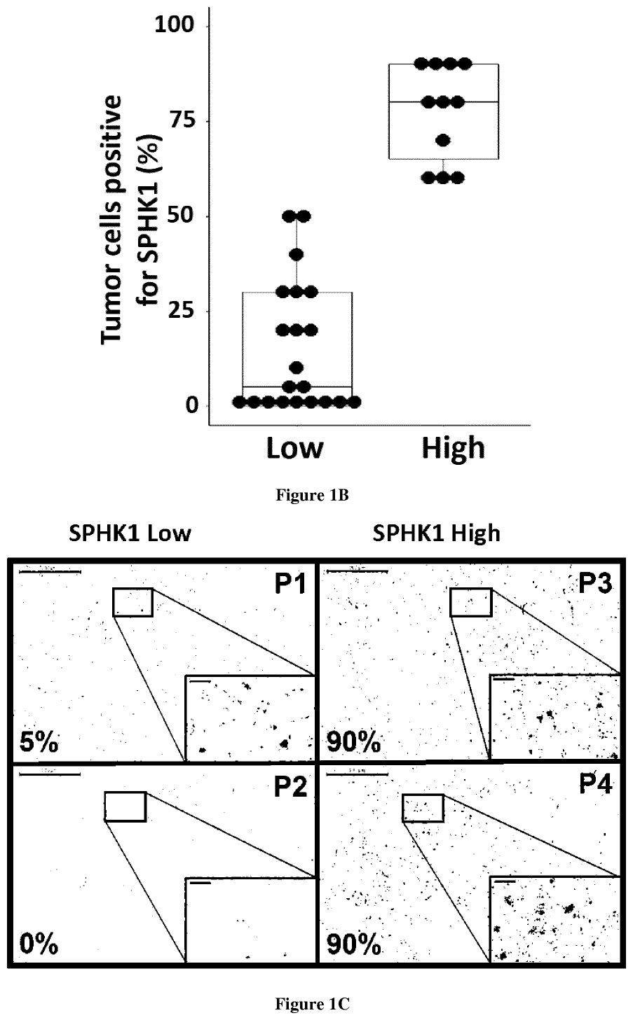

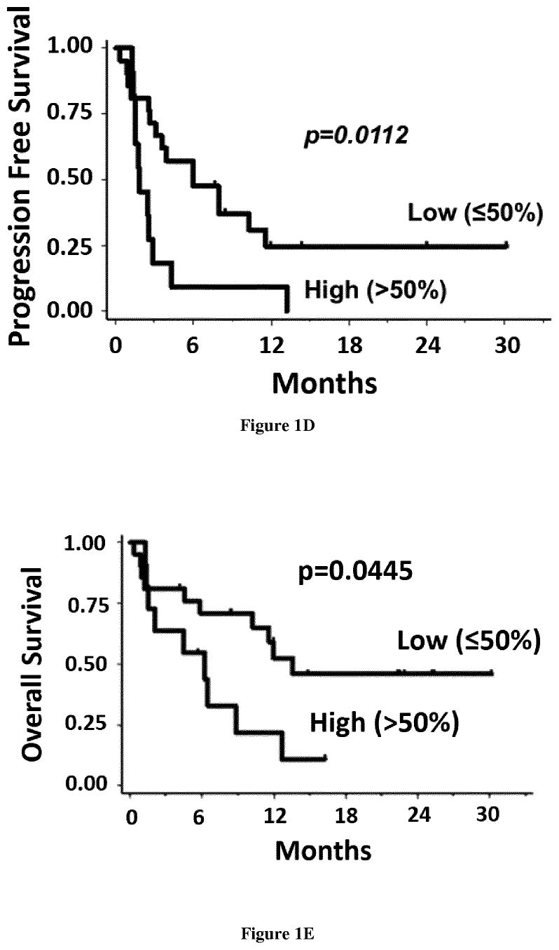

Analysis of two different cohorts from the Oncomine database indicated that SPHK1 (encoding SK1) transcript levels were higher in human primary melanomas as compared to nevi (FIG. 1A, left panel); SPHK1 expression was further increased in metastatic melanomas (FIG. 1A, right panel), suggesting that SPHK1 expression might be associated with melanoma progression.

In order to evaluate whether SPHK1 expression is related to the clinical outcome of advanced melanoma patients receiving anti-PD-1 therapy (Table B), we analyzed SPHK1 mRNA in tumor biopsies by in situ hybridization using the RNAscope technology. According to the distribution of the percentage of tumor cells positive for SPHK1, two groups of patients named SPHK1 Low (<50%) and SPHK1 High (>50%) were defined (FIG. 1B). FIG. 2C shows representative SPHK1 staining for these two groups. Kaplan-Meier analysis revealed that patients with low SPHK1 expression had longer PFS and OS than those with high SPHK1 expression (p=0.0112 and p=0.0445, respectively) (FIGS. 1D and E). These findings support the hypothesis that SPHK1 expression represents a potential biomarker to predict tumor progression and resistance to anti-PD1 in metastatic melanoma patients.

TABLE B
Clinical characteristics of the anti-PD-1 cohort. Continuous variables
were presented as median with range (min-max) and categorical
variables were summarized by frequencies and percentages.

| | | |
|---|---|---|
| Total | SK1low (<=50%) | SK1high (>50%) |
| N = 32 | N = 21 | N = 11 |

Gender(n = 32)

| | | | | | | |
|---|---|---|---|---|---|---|
| Male | 21 | (65.6%) | 15 | (71.4%) | 6 | (54.5%) |
| Female | 11 | (34.4%) | 6 | (28.6%) | 5 | (45.5%) |

Age at treatment initiation (n = 32)

| | | | | | | |
|---|---|---|---|---|---|---|
| <=65 years | 13 | (40.6%) | 8 | (38.1%) | 5 | (45.5%) |
| >65 years | 19 | (59.4%) | 13 | (61.9%) | 6 | (54.5%) |

Who Performance Status (n = 31)

| | | | | | | |
|---|---|---|---|---|---|---|
| 0 | 20 | (64.5%) | 13 | (65.0%) | 7 | (63.6%) |
| 1 | 11 | (35.5%) | 7 | (35.0%) | 4 | (36.4%) |

| | | | |
|---|---|---|---|
| Missing | 1 | 1 | 0 |

Stage (n = 32)

| | | | | | | |
|---|---|---|---|---|---|---|
| IIIc | 5 | (15.6%) | 5 | (23.8%) | 0 | (0.0%) |
| IV | 5 | (15.6%) | 4 | (19.0%) | 1 | (9.1%) |
| IVa | 5 | (15.6%) | 4 | (19.0%) | 1 | (9.1%) |
| IVb | 5 | (15.6%) | 1 | (4.8%) | 4 | (36.4%) |
| IVc | 12 | (37.5%) | 7 | (33.3%) | 5 | (45.5%) |

Histological subtype (n = 32)

| | | | | | | |
|---|---|---|---|---|---|---|
| Mucosal | 1 | (3.1%) | 1 | (4.8%) | 0 | (0.0%) |
| Cutaneous | 30 | (93.8%) | 20 | (95.2%) | 10 | (90.9%) |
| Other | 1 | (3.1%) | 0 | (0.0%) | 1 | (9.1%) |

BRAF(n = 31)

| | | | | | | |
|---|---|---|---|---|---|---|
| No | 22 | (71.0%) | 12 | (60.0%) | 10 | (90.9%) |
| Yes | 9 | (29.0%) | 8 | (40.0%) | 1 | (9.1%) |

| | | | |
|---|---|---|---|
| Missing | 1 | 1 | 0 |

NRAS(n = 27)

| | | | | | | |
|---|---|---|---|---|---|---|
| No | 17 | (63.0%) | 9 | (56.3%) | 8 | (72.7%) |
| Yes | 10 | (37.0%) | 7 | (43.8%) | 3 | (27.3%) |

| | | | |
|---|---|---|---|
| Missing | 5 | 5 | 0 |

Treatment line (n=32)

| | | | | | | |
|---|---|---|---|---|---|---|
| 1 | 23 | (71.9%) | 14 | (66.7%) | 9 | (81.8%) |
| 2 | 6 | (18.8%) | 5 | (23.8%) | 1 | (9.1%) |
| 3 | 3 | (9.4%) | 2 | (9.5%) | 1 | (9.1%) |

Treatment line (n = 32)

| | | | | | | |
|---|---|---|---|---|---|---|
| <2 | 23 | (71.9%) | 14 | (66.7%) | 9 | (81.8%) |
| >=2 | 9 | (28.1%) | 7 | (33.3%) | 2 | (18.2%) |

DCI(n = 32)

| | | | | | | |
|---|---|---|---|---|---|---|
| Pembrolizumab | 22 | (68.8%) | 13 | (61.9%) | 9 | (81.8%) |
| Nivolumab | 10 | (31.3%) | 8 | (38.1%) | 2 | (18.2%) |

### REFERENCES

Throughout this application, various references describe the state of the art to which this invention pertains. The disclosures of these references are hereby incorporated by reference into the present disclosure.
8. V. Albinet et al., Dual role of sphingosine kinase-1 in promoting the differentiation of dermal fibroblasts and the dissemination of melanoma cells. Oncogene 33, 3364-3373 (2014).
9. M. Mrad et al., Downregulation of sphingosine kinase-1 induces protective tumor immunity by promoting Ml macrophage response in melanoma. Oncotarget 7, 71873-71886 (2016).
10. N. J. Pyne, J. Ohotski, R. Bittman, S. Pyne, The role of sphingosine 1-phosphate in inflammation and cancer. Adv Biol Regul 54, 121-129 (2014).
11. N. J. Pyne, S. Pyne, Sphingosine 1-phosphate and cancer. Nat Rev Cancer 10, 489-503 (2010).
12. C. S. Garris, V. A. Blaho, T. Hla, M. H. Han, Sphingosine-1-phosphate receptor 1 signalling in T cells: trafficking and beyond. Immunology 142, 347-353 (2014).
13. S. Spiegel, S. Milstien, The outs and the ins of sphingosine-1-phosphate in immunity. Nat Rev Immunol 11, 403-415 (2011).

1. A method for determining whether a patient suffering from a cancer will achieve a response with an immune checkpoint inhibitor comprising i) determining the expression level of SK1 in a tumor sample obtained from the patient, ii) comparing the expression level determined at step i) with a predetermined reference value and iii) concluding that the patient will not achieve a response when the level determined at step i) is higher than the predetermined reference value or concluding that the patient will achieve a response when the level determined at step i) is lower than the predetermined reference value.

2. The method of claim 1 wherein the patient suffer from a cancer selected from the group consisting of neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

3. The method of claim 1 wherein the patient suffers from melanoma.

4. The method of claim 1 wherein the patient suffers from a metastatic melanoma.

5. The method of claim 1 wherein the immune checkpoint inhibitor is an antibody selected from the group consisting of anti-CTLA4 antibodies, anti-PD1 antibodies, anti-PDL1 antibodies, anti-TIM-3 antibodies, anti-LAG3 antibodies, anti-B7H3 antibodies, anti-B7H4 antibodies, anti-BTLA antibodies, and anti-B7H6 antibodies.

6. The method of claim 1 wherein, the tumor sample is from a tumor resected from the patient.

7. The method of claim 1 wherein the tumor sample is from a biopsy performed in a primary tumor of the patient or in a metastatic sample distant from the primary tumor of the patient.

8. The method of claim 1 wherein the tumor sample is a sample of circulating tumor cells.

9. The method of claim 1 wherein the expression level of SK1 is determined by immunodetection.

10. The method of claim 1 wherein the expression level of SK1 is determined by detecting the quantity of mRNA encoding for SK1.

11. A method of treating a patient suffering from a cancer comprising i) determining the expression level of SK1 in a tumor sample obtained from the patient, ii) comparing the expression level determined at step i) with a predetermined reference value and (iii) administering to said patient a therapeutically effective amount of a SK1 inhibitor when the level determined at step i) is higher than the predetermined reference value.

12. The method of claim 11 wherein the SK1 inhibitor is administered with an immune checkpoint inhibitor as a combined preparation.

13. The method of claim 9, wherein the immunodetection is performed by immunohistochemistry (IHC) or immunofluorescence.


SEQUENCE LISTING (2 sequences)

SEQ ID NO: 1 - protein, 384 aa, Homo sapiens
MDPAGGPRGVLPRPCRVLVLLNPRGGKGKALQLFRSHVQPLLAEAEISFTLMLTERRNHARELVRSEELGRWDALVVMSGDGLMHEVVNGLMERPDWETAIQKPLCSLPAGSGNALAASLNHYAGYEQVTNEDLLTNCTLLLCRRLLSPMNLLSLHTASGLRLFSVLSLAWGFIADVDLESEKYRRLGEMRFTLGTFLRLAALRTYRGRLAYLPVGRVGSKTPASPVVVQQGPVDAHLVPLEEPVPSHWTVVPDEDFVLVLALLHSHLGSEMFAAPMGRCAAGVMHLFYVRAGVSRAMLLRLFLAMEKGRHMEYECPYLVYVPVVAFRLEPKDGKGVFAVDGELMVSEAVQGQVHPNYFWMVSGCVEPPPSWKPQQMPPPEEPL

SEQ ID NO: 2 - DNA, 1839 bp, Homo sapiens
agtgccctccccgctccgcggcgccggctgcgaagttgagcgaaaagtttgaggccggagggagcgaggccggggagtccgctccagcggggcgctccagtccctcagacgtgggctgagcttgggacgagctgcgttccgccccaggccactgtagggaacggcggtggcgcctccccagcaaaccggaccgactgggtccagccgccgcagggaatgacgccggtgctcctgcagccacggctccgggcggggaaggcgagccccacagccggccctgcgacgcccgcctgggcagcaccgataaggagctgaaggcaggagccgccgccacgggcagcgcccccacagcgccagggaccccctggcagcgggagccgcgggtcgaggttatggatccagcgggcggcccccggggcgtgctcccgcggccctgccgcgtgctggtgctgctgaacccgcgcggcggcaagggcaaggccttgcagctcttccggagtcacgtgcagccccttttggctgaggctgaaatctccttcacgctgatgctcactgagcggcggaaccacgcgcgggagctggtgcggtcggaggagctgggccgctgggacgctctggtggtcatgtctggagacgggctgatgcacgaggtggtgaacgggctcatggagcggcctgactgggagaccgccatccagaagcccctgtgtagcctcccagcaggctctggcaacgcgctggcagcttccttgaaccattatgctggctatgagcaggtcaccaatgaagacctcctgaccaactgcacgctattgctgtgccgccggctgctgtcacccatgaacctgctgtctctgcacacggcttcggggctgcgcctcttctctgtgctcagcctggcctggggcttcattgctgatgtggacctagagagtgagaagtatcggcgtctgggggagatgcgcttcactctgggcaccttcctgcgtctggcagccctgcgcacctaccgcggccgactggcctacctccctgtaggaagagtgggttccaagacacctgcctcccccgttgtggtccagcagggcccggtagatgcacaccttgtgccactggaggagccagtgccctctcactggacagtggtgcccgacgaggactttgtgctagtcctggcactgctgcactcgcacctgggcagtgagatgtttgctgcacccatgggccgctgtgcagctggcgtcatgcatctgttctacgtgcgggcgggagtgtctcgtgccatgctgctgcgcctcttcctggccatggagaagggcaggcatatggagtatgaatgcccctacttggtatatgtgcccgtggtcgccttccgcttggagcccaaggatgggaaaggtgtgtttgcagtggatggggaattgatggttagcgaggccgtgcagggccaggtgcacccaaactacttctggatggtcagcggttgcgtggagcccccgcccagctggaagccccagcagatgccaccgccagaagagcccttatgacccctgggccgcgctgtgccttagtgtctacttgcaggacccttcctccttccctagggctgcagggcctgtccacagctcctgtgggggtggaggagactcctctggagaagggtgagaaggtggaggctatgctttggggggacaggccagaatgaagtcctgggtcaggagcccagctggctgggcccagctgcctatgtaaggccttctagtttgttctgagacccccaccccacgaaccaaatccaaataaagtgacattcccagcctgaaaaaaaaaaaaaaaaaa